(19) United States
(12) Patent Application Publication
(10) Pub. No.: US 2013/0045943 A1
(43) Pub. Date: Feb. 21, 2013

(54) A3AR AGONISTS FOR THE TREATMENT OF UVEITIS

(71) Applicants: Rachel Caspi, Bethesda, MD (US); Pnina Fishman, Herzliya, IL (US)

(72) Inventors: Rachel Caspi, Bethesda, MD (US); Pnina Fishman, Herzliya, IL (US)

(73) Assignee: THE GOVERNMENT OF THE UNITED STATES OF AMERICA AS REPRESENTED BY THE SECRETARY OF THE DEPARTMENT OF, BETHESDA, MD (US)

(21) Appl. No.: 13/581,884

(22) PCT Filed: Feb. 27, 2011
(86) PCT No.: PCT/IL11/00193

§ 371 Date: Nov. 7, 2012

Related U.S. Application Data
(60) Provisional application No. 61/310,043, filed on Mar. 3, 2010.

(51) Int. Cl. A61K 31/7076; A61P 27/02

(52) U.S. Cl. 514/46

(57) ABSTRACT

The present disclosure relates to the use of an A₃AR agonist, such as IB-MECA, for the treatment or prevention of uveitis in a subject, as well as to methods for such treatment and pharmaceutical compositions comprising an amount of IB-MECA effective to treat uveitis.

### FIELD OF THE INVENTION

This invention relates to methods and compositions for the treatment of uveitis.

### BACKGROUND OF THE INVENTION

Uveitis specifically refers to inflammation of the middle layer of the eye (the “uvea”), providing most of the blood supply to the retina, but in common usage may refer to any inflammatory process involving the interior of the eye, with inflammation specifically of the uvea termed iridocyclitis.

Uveitis is typically caused by autoimmune disorders, infection or exposure to toxins. Symptoms of uveitis consist of redness of the eye, blurred vision, sensitivity to light (photophobia), dark, floating spots in the vision and eye pain.

Uveitis is estimated to be responsible for approximately 10% of the blindness in the United States. Uveitis requires an urgent referral and thorough examination by an ophthalmologist, along with urgent treatment to control the inflammation. The prognosis is generally good for those who receive prompt diagnosis and treatment, but serious complication (including cataracts, glaucoma, fluids within the retina, retinal detachment and vision loss, band keratopathy, retinal edema and permanent vision loss) may result if left untreated. The type of uveitis, as well as its severity, duration, and responsiveness to treatment or any associated illnesses, all factor in to the outlook.

Eye drops, especially glucocorticoid steroids (e.g. prednisolone acetate) and pupil dilators, or oral therapy with prednisolone tablets are medications used to reduce the inflammation and the pain in uveitis. In addition topical cycloplegics, such as atropine or homatropine, may be used. For deeper inflammation, oral medication or periocular injections of the steroids or immuno-suppressants are used Also, antimetabolite medications, such as methotrexate are often used for recalcitrant or more aggressive cases of uveitis. [Nussenblatt R B, Whitcup S M. (2004) Uveitis: Fundamentals and Clinical Practice (3rd edn), Mosby/Elsevier; 2004; Gery I, Nussenblatt R B, Chan C C, Caspi R R. Autoimmune diseases of the eye. The molecular pathology of autoimmune diseases. 2nd ed. New York, N.Y.: Taylor and Francis; 2002:978-98].

An acceptable experimental autoimmune eveitis (EAU) model is an organ-specific, T-cell-mediated autoimmune disease that targets the neural retina and related tissues and is considered a model of autoimmune uvetitis in humans. It is induced by immunization of rats or mice with retinal antigens. The pathology of EAU closely resembles human uveitic diseases of a putative autoimmune nature in which patients display immunological responses to retinal antigens [Caspi R R, Silver P B, Luger D, Tang J, Cortes L M, Pennesi G, Mattapallil M J, Chan C C. Mouse models of experimental autoimmune uveitis. Ophthalmic Res. 2008; 40:169-74; Smith J R, Hart P H, Williams K A. Basic pathogenic mechanisms operating in experimental models of acute anterior uveitis. Immunol. Cell Biol. 1998; 76, 497-512; Caspi R R. in Cohen, I. R. and Miller, A. (eds.), Animal Models for Autoimmune Diseases: A Guidebook, Academic Press p. 57-81. 1994].

### SUMMARY OF THE INVENTION

The present invention is based on the finding that N⁶-(3-iodobenzyl)-2-methylamino-9-[5-(methylamido)-β-D-ribofuranosyl]-adenine (herein referred to by the abbreviation IB-MECA) was effective in the following:
it inhibited development of experimental autoimmune uveitis in animal model;
it decreased the histopathological score of experimental autoimmune eveitis (EAU);
it ameliorated antigen-specific T cell response.

Based on these findings it has been envisaged that the A₃ adenosine receptor (A₃AR) agonist, IB-MECA, serving as an exemplary A₃AR agonist, may be used for the treatment or prevention of uveitis.

Thus, in accordance with a first of its aspects the present invention provides the use of an A₃AR agonist for the treatment or prevention of uveitis.

In accordance with a second aspect, the present invention provides a method for the treatment of uveitis comprising administering a subject an amount of A₃AR agonist, the amount being effective to treat or prevent uveitis.

In yet a third aspect, the present invention provides a pharmaceutical composition for treating uveitis comprising as active ingredient an amount of A₃AR agonist and a physiologically acceptable carrier, the amount of said A₃AR agonist being effective to treat said uveitis.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a bar graph showing that IB-MECA, identified by the code name CF101, inhibited the development of EAU.

FIG. 2 is a bar graph showing that IB-MECA, identified by the code name CF101, decreased the histopathological score of EAU.

FIG. 3 is a bar graph showing that IB-MECA, identified by the code name CF101, ameliorates antigen-specific T cell response

### DETAILED DESCRIPTION OF EMBODIMENTS

As appreciated, while the invention is described in the following detailed description with reference to a method of treatment of uveitis making use of A₃AR agonist, it is to be understood to also encompass the use of A₃AR agonist for treating uveitis as well any pharmaceutical composition comprising the A₃AR for said treatment.

In the context of the present invention the term “uveitis” denotes an inflammation of the interior segment of the eye, particularly, inflammation of the middle layer (uvea) of the eye. More specifically, uveitis includes, Anterior uveitis, being an inflammation of the front part of the uveal tract; including inflammation of the iris (iritis) and inflammation of the iris and the ciliary body (iridocyclitis); Intermediate uveitis (peripheral uveitis or chronic cyclitis) being inflammation in the vitreous; and Posterior uveitis, being an inflammation of the part of the uveal tract behind the lens of the eye; inflammation of the choroid (choroiditis) and inflammation of the choroid and retina (chorioretinitis); as well as panuveitis or diffuse uveitis being uveitis that affects the entire uveal tract.

The terms “treating” or “treatment”, and the like are used herein to refer to obtaining a desired pharmacological and physiological effect. The effect may be therapeutic in terms of ameliorating or reducing inflammatory response in the interior segment of the eye and/or prophylactic, in terms of preventing or partially preventing the development of inflammation in the interior segment of the eye in any subject who may be predisposed to develop inflammation in the interior portion of the eye e.g. as a result of one or more of trauma to the interior eye, ocular and systemic infection (viral, bacterial, Parasitic uveitis), and systemic autoimmune disorder. The treatment is to be understood to encompass any treatment of a disease in a mammal, particularly a human.

The term “A₃ adenosine receptor agonist” (A₃AR agonist) in the context of the present invention refers to any molecule capable of specifically binding to the A₃ adenosine receptor (A₃AR), thereby fully or partially activating said receptor. The A₃AR agonist is thus a molecule that exerts its prime effect through the binding and activation of the A₃AR. This means that at the doses it is being administered it essentially binds to and activates only the A₃AR. In a preferred embodiment, an A₃AR agonist has a binding affinity (Kᵢ) to the human A₃AR in the range of less than 100 nM, typically less than 50 nM, preferably less than 20 nM, more preferably less than 10 nM and ideally less than 5 nM. Particularly preferred are A₃AR agonists that have a Kᵢ to the human A₃R of less than 2 nM and desirably less than 1 nM.

However, it should be understood that some A₃AR agonists can also interact with and activate other receptors, however, with lower affinities (namely a higher Ki). A molecule will be considered an A₃AR agonist in the context of the invention (namely a molecule that exerts its prime effect through the binding and activation A₃AR) if its affinity to the A₃AR is at least 3 times (i.e. its Ki to the A₃AR is at least 3 times lower), preferably 10 times, desirably 20 times and most preferably at least 50 times larger than the affinity to any other of the adenosine receptors (i.e. A₁, A₂ₐ and A_{2b}).

The affinity of an A₃AR agonist to the human A₃AR as well as its relative affinity to the other human adenosine receptors can be determined by a number of assays, such as a binding assay. Examples of binding assays include providing membranes containing a receptor and measuring the ability of the A₃AR agonist to displace a bound radioactive agonist; utilizing cells that display the respective human adenosine receptor and measuring, in a functional assay, the ability of the A₃AR agonist to activate or deactivate, as the case may be, downstream signaling events such as the effect on adenylate cyclase measured through increase or decrease of the cAMP level; etc. Clearly, if the administered level of an A₃AR agonist is increased such that its blood level reaches a level approaching that of the Ki of the A₁, A₂ₐ and A_{2b} adenosine receptors, activation of these receptors may occur following such administration, in addition to activation of the A₃AR. An A₃AR agonist is thus preferably administered at a dose such that the blood level is such so that essentially only the A₃AR will be activated.

The characteristic of some adenosine A₃AR agonists and methods of their preparation are described in detail in, inter alia, U.S. Pat. No. 5,688,774; U.S. Pat. No. 5,773,423, U.S. Pat. No. 5,573,772, U.S. Pat. No. 5,443,836, U.S. Pat. No. 6,048,865, WO 95/02604, WO 99/20284, WO 99/06053, WO 97/27173 and WO 01/19360, all of which are incorporated herein by reference.

According to an embodiment of the invention, the A₃AR agonist is a compound that exerts its prime effect through the binding and activation of the adenosine A₃AR and is a purine derivative falling within the scope of the general formula (I):

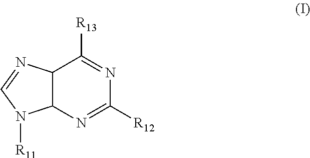

wherein,
R₁₁ represents an alkyl, hydroxyalkyl, carboxyalkyl or cyanoalkyl or a group of the following general formula (II):

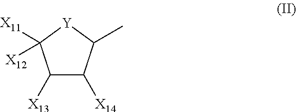

in which:
Y represents oxygen, sulfur or CH₂;
X₁₁ represents H, alkyl, R^{e}R^{f}NC(═O)— or HOR^{g}—, wherein
R^{e} and R^{f} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{g} is selected from the group consisting of alkyl, amino, haloalkyl, aminoalkyl, BOC-aminoalkyl, and cycloalkyl;
X₁₂ is H, hydroxyl, alkylamino, alkylamido or hydroxyalkyl;
X₁₃ and X₁₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₁₃ and X₁₄ are oxygens connected to >C═S to form a 5-membered ring, or X₁₂ and X₁₃ form the ring of formula (III):

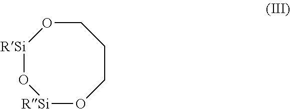

where R′ and R″ represent independently an alkyl group;

R₁₂ is selected from the group consisting of hydrogen, halo, alkylether, amino, hydrazido, alkylamino, alkoxy, thioalkoxy, pyridylthio, alkenyl; alkynyl, thio, and alkylthio; and

R₁₃ is a group of the formula —NR₁₅R₁₆ wherein

R₁₅ is a hydrogen atom or a group selected from alkyl, substituted alkyl or aryl-NH—C(Z)—, with Z being O, S, or NR^{a} with R^{e} having the above meanings; wherein when R₁₅ is hydrogen than

R₁₆ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₁₆ is a group of the following formula:

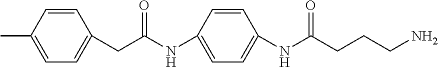 [SMILES: Cc1ccc(CC(=O)Nc2ccc(NC(=O)CCCN)cc2)cc1]

or when R₁₅ is an alkyl or aryl-NH—C(Z)—, then, R₁₆ is selected from the group consisting of heteroaryl-NR^{a}—C(Z)—, heteroaryl-C(Z)—, alkaryl-NR^{a}—C(Z)—, alkaryl-C(Z)—, aryl-NR—C(Z)— and aryl-C(Z)—; Z representing an oxygen, sulfor or amine.

Exemplary A₃AR agonist (disclosed in U.S. Pat. No. 5,688,774 at column 4, lines 67-column 6, line 16; column 5, lines 40-45; column 6, lines 21-42; column 7, lines 1-11; column 7, lines 34-36; and column 7, lines 60-61):
N⁶-(3-iodobenzyl)-9-methyladenine;
N⁶-(3-iodobenzyl)-9-hydroxyethyladenine;
R—N⁶-(3-iodobenzyl)-9-(2,3-dihydroxypropyl)adenine;
S—N⁶-(3-iodobenzyl)-9-(2,3-dihydroxypropyl)adenine;
N⁶-(3-iodobenzyladenin-9-yl)acetic acid;
N⁶-(3-iodobenzyl)-9-(3-cyanopropyl)adenine;
2-chloro-N⁶-(3-iodobenzyl)-9-methyladenine;
2-amino-N⁶-(3-iodobenzyl)-9-methyladenine;
2-hydrazido-N⁶-(3-iodobenzyl)-9-methyladenine;
N⁶-(3-iodobenzyl)-2-methylamino-9-methyladenine;
2-dimethylamino-N⁶-(3-iodobenzyl)-9-methyladenine;
N⁶-(3-iodobenzyl)-9-methyl-2-propylaminoadenine;
2-hexylamino-N⁶-(3-iodobenzyl)-9-methyladenine;
N⁶-(3-iodobenzyl)-2-methoxy-9-methyladenine;
N⁶-(3-iodobenzyl)-9-methyl-2-methylthioadenine;
N⁶-(3-iodobenzyl)-9-methyl-2-(4-pyridylthio)adenine;
(1S, 2R, 3S, 4R)-4-(6-amino-2-phenylethylamino-9H-purin-9-yl)cyclopentane-1,2,3-triol;
(1S, 2R, 3S, 4R)-4-(6-amino-2-chloro-9H-purin-9-yl)cyclopentane-1,2,3-triol;
(±)-9-[2α,3α-dihydroxy-4β-(N-methylcarbamoyl)cyclopent-1β-yl)]-N⁶-(3-iodobenzyl)-adenine;
2-chloro-9-(2′-amino-2′,3′-dideoxy-β-D-5′-methyl-arabino-furonamido)-N⁶-(3-iodobenzyl)adenine;
2-chloro-9-(2′,3′-dideoxy-2′-fluoro-β-D-5′-methyl-arabino furonamido)-N⁶-(3-iodobenzyl)adenine;
9-(2-acetyl-3-deoxy-β-D-5-methyl-ribofuronamido)-2-chloro-N⁶(3-iodobenzyl)adenine;
2-chloro-9-(3-deoxy-2-methanesulfonyl-β-D-5-methyl-ribofuronamido)-N⁶-(3-iodobenzyl)adenine;
2-chloro-9-(3-deoxy-β-D-5-methyl-ribofuronamido)-N⁶-(3-iodobenzyl)adenine;
2-chloro-9-(3,5-1,1,3,3-tetraisopropyldisiloxyl-β-D-5-ribofuranosyl)-N⁶-(3-iodobenzyl)adenine;
2-chloro-9-(2′,3′-O-thiocarbonyl-β-D-5-methyl-ribofuronamido)-N⁶-(3-iodobenzyl)adenine;
9-(2-phenoxythiocarbonyl-3-deoxy-β-D-5-methyl-ribofuronamido)-2-chloro-N⁶-(3-iodobenzyl)adenine;
1-(6-benzylamino-9H-purin-9-yl)-1-deoxy-N,4-dimethyl-β-D-ribofuranosiduronamide;
2-chloro-9-(2,3-dideoxy-β-D-5-methyl-ribofuronamido)-N⁶benzyladenine;
2-chloro-9-(2′-azido-2′,3′-dideoxy-β-D-5′-methyl-arabino-furonamido)-N⁶-benzyladenine;
2-chloro-9-(β-D-erythrofuranoside)-N⁶-(3-iodobenzyl)adenine;
N⁶-(benzodioxanemethyl)adenosine;
1-(6-furfurylamino-9H-purin-9-yl)-1-deoxy-N-methyl-β-D-ribofuranosiduronamide;
N⁶-[3-(L-prolylamino)benzyl]adenosine-5′-N-methyluronamide;
N⁶-[3-(β-alanylamino)benzyl]adenosine-5′-N-methyluronamide;
N⁶-[3-(N-T-Boc-β-alanylamino)benzyl]adenosine-5′-N-methyluronamide
6-(N′-phenylhydrazinyl)purine-9-β-ribofuranoside-5′-N-methyluronamide;
6-(O-phenylhydroxylamino)purine-9-β-ribofuranoside-5′-N-methyluronamide;
9-(β-D-2′,3′-dideoxyerythrofuranosyl)-N⁶-[(3-β-alanylamino)benzyl]adenosine;
9-(β-D-erythrofuranoside)-2-methylamino-N⁶-(3-iodobenzyl)adenine;
2-chloro-N-(3-iodobenzyl)-9-(2-tetrahydrofuryl)-9H-purin-6-amine;
2-chloro-(2′-deoxy-6′-thio-L-arabinosyl)adenine; and
2-chloro-(6′-thio-L-arabinosyl)adenine.

Other exemplary A₃AR agonists, disclosed in U.S. Pat. No. 5,773,423, are compounds of the formula:

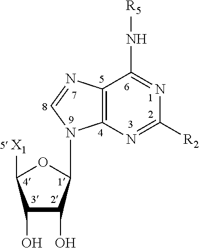

wherein

X₁ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;

R₂ is selected from the group consisting of hydrogen, halo, C₁-C₁₀ alkyoxy, amino, C₂-C₁₀ alkenyl, and C₂-C₁₀ alkynyl; and

R₅ is selected from the group consisting of R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo.

More specific compounds include those of the above formula wherein R^{a} and R^{b} may be the same or different and are selected from the group consisting of hydrogen and C₁-C₁₀ alkyl, particularly when R₂ is hydrogen or halo, especially hydrogen.

Additional specific compounds are those compounds wherein R^{a} is hydrogen and R₂ is hydrogen, particularly when R₅ is unsubstituted benzyl.

More specific compounds are such compounds wherein R^{b} is a C₁-C₁₀ alkyl or C₃-C₁₀ cycloalkyl, particularly a C₁-C₁₀ alkyl, and more particularly methyl.

Especially specific are those compounds where R^{a} is hydrogen, R^{b} is C₁-C₁₀ alkyl or C₃-C₁₀ cycloalkyl, and R₅ is R- or S-1-phenylethyl or a benzyl substituted in one or more positions with a substituent selected from the group consisting of halo, amino, acetamido, C₁-C₁₀ haloalkyl, and sulfo, where the sulfo derivative is a salt, such as a triethylammonium salt.

An example of an especially preferred compound is IB-MECA (disclosed in U.S. Pat. No. 5,773,423). In addition, those compounds in which R₂ is a C₂-C₁₀ alkenylene of the formula R^{d}—C═C— where R^{d} is a C₁-C₈ alkyl are also particularly noted in U.S. Pat. No. 5,773,423.

Also specific are those compounds wherein R₂ is other than hydrogen, particularly those wherein R₂ is halo, C₁-C₁₀ alkylamino, or C₁-C₁₀ alkylthio, and, more preferably, when additionally R^{a} is hydrogen, R^{b} is a C₁-C₁₀ alkyl, and/or R₅ is a substituted benzyl.

Further exemplary A₃AR agonists disclosed in U.S. Pat. No. 5,773,423 are modified xanthine-7-ribosides having the formula:

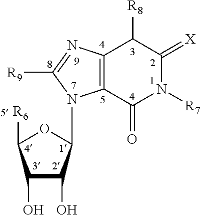

wherein

X is O;

R₆ is R^{a}R^{b}NC(═O), wherein R^{a} and R^{b} may be the same or different and are selected from the group consisting of hydrogen, C₁-C₁₀ alkyl, amino, C₁-C₁₀ haloalkyl, C₁-C₁₀ aminoalkyl, and C₃-C₁₀ cycloalkyl;

R₇ and R₈ may be the same or different and are selected from the group consisting of C₁-C₁₀ alkyl, R- and S-1-phenylethyl, an unsubstituted benzyl group, and a benzyl group substituted in one or more positions with a substituent selected from the group consisting of C₁-C₁₀ alkyl, amino, halo, C₁-C₁₀ haloalkyl, nitro, hydroxy, acetamido, C₁-C₁₀ alkoxy, and sulfo; and

R₉ is selected from the group consisting of halo, benzyl, phenyl, and C₃-C₁₀ cycloalkyl.

WO 99/06053 discloses in examples 19-33 compounds selected from:

N⁶-(4-biphenyl-carbonylamino)-adenosine-5′-N-ethyluronamide;

N⁶-(2,4-dichlorobenzyl-carbonylamino)-adenosine-5′-N-ethyluronamide;

N⁶-(4-methoxyphenyl-carbonylamino)-adenosine-5′-N-ethyluronamide;

N⁶-(4-chlorophenyl-carbonylamino)-adenosine-5′-N-ethyluronamide;

N⁶-(phenyl-carbonylamino)-adenosine-5′-N-ethyluronamide;

N⁶-(benzylcarbamoylamino)-adenosine-5′-N-ethyluronamide;

N⁶-(4-sulfonamido-phenylcarbamoyl)-adenosine-5′-N-ethyluronamide;

N⁶-(4-acetyl-phenylcarbamoyl)-adenosine-5′-N-ethyluronamide;

N⁶-((R)-α-phenylethylcarbamoyl)-adenosine-5′-N-ethyluronamide;

N⁶-((S)-α-phenylethylcarbamoyl)-adenosine-5′-N-ethyluronamide;

N⁶-(5-methyl-isoxazol-3-yl-carbamoyl)-adenosine-5′-N-ethyluronamide;

N⁶-(1,3,4-thiadiazol-2-yl-carbamoyl)-adenosine-5′-N-ethyluronamide;

N⁶-(4-n-propoxy-phenylcarbamoyl)-adenosine-5′-N-ethyluronamide;

N⁶-bis-(4-nitrophenylcarbamoyl)-adenosine-5′-N-ethyluronamide; and

N⁶-bis-(5-chloro-pyridin-2-yl-carbamoyl)-adenosine-5′-N-ethyluronamide.

More specifically disclosed compounds include:

2-chloro-N⁶-(3-iodobenzyl)-9-[5-(methylamido)-β-D-ribofuranosyl]-adenine also known as 2-chloro-N⁶-(3-iodobenzyl)-adenosine-5′-N-methyluronamide or by the abbreviation Cl-IB-MECA;

N⁶-(3-iodobenzyl)-2-methylamino-9-[5-(methylamido)-β-D-ribofuranosyl]-adenine, also known as N⁶-(3-iodobenzyl)-adenosine-5′-N-methyluronamide or known as 1-Deoxy-1-[6-[[(3-iodophenyl)methyl]amino]-9H-purine-9-yl]-N-methyl-D-ribofuranuronamide or by the abbreviation IB-MECA;

N⁶-2-(4-aminophenyl)ethyladenosine (APNEA);

N⁶-(4-amino-3-iodobenzyl)adenosine-5′-(N-methyluronamide) (AB-MECA).

IB-MECA is the most preferred compound in accordance with the invention.

Also encompassed by the invention are any physiologically acceptable salt of the above defined compounds. When referring to “physiologically acceptable salts” of the compounds employed by the present invention it is meant any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the compounds of this invention with a suitable organic or inorganic acid. The acid addition salts are those which retain the biological effectiveness and qualitative properties of the free bases and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g. p-toluenesulphonic, acids.

The terms “effective amount” or “amount effective to” in the context of the present invention refer to an amount of A₃AR agonist which prevents or treat uveitis, in subjects predisposed to develop or who have already developed uveitis. The “effective amount” can be readily determined, in accordance with the invention, by administering to a plurality of tested subjects various amounts of the A₃AR agonist and then plotting the physiological response (for example an integrated “SS index” combining several of the therapeutically beneficial effects) as a function of the amount. Alternatively, the effective amount may also be determined, at times, through experiments performed in appropriate animal models and then extrapolating to human beings using one of a plurality of conversion methods; or by measuring the plasma concentration or the area under the curve (AUC) of the plasma concentration over time and calculating the effective dose so as to yield a comparable plasma concentration or AUC. As known, the effective amount may depend on a variety of factors such as mode of administration (for example, oral administration may require a higher dose to achieve a given plasma level or AUC than an intravenous administration); the age, weight, body surface area, gender, health condition and genetic factors of the subject; other administered drugs; etc.

In the following, unless otherwise indicated, dosages are indicated in weight/Kg, meaning weight of administered A₃AR agonist (e.g. IB-MECA) per kilogram of body weight of the treated subject in each administration. For example, mg/Kg and microgram/Kg denote, respectively, milligrams of administered agent and micrograms of administered agent per kilogram of body weight of the treated subject.

The effective amount is preferably less than about 1 mg/kg body weight, particularly less than about 500 μg/kg or even less than about 200 μg/kg body weight or at times less than about 100 μg/kg body weight or even less than about less than 50 μg/kg body weight. With respect to IB-MECA, the effective amount is preferably less than 5 mg each dose, for once daily administration (namely a dose less than about 70 μg/kg body weight, assuming an average individual weight of about 70 kg), and less than about 4 mg each dose (i.e. less than about 57 μg/kg body weight), for twice daily administration. The dose of IB-MECA is more preferably less than about 2 mg each dose and typically between about 0.1-1 mg each dose, for either once or twice daily administration (the corresponding dosages in weight per body weight being about 29 μg/kg and about 1.5-15 μg/kg body weight, respectively).

The administration of the A₃AR agonist to an individual may be together with a pharmaceutically acceptable carrier to form a dosage form suitable for a specific mode of administration. The dosage form is thus the physical form of A₃AR agonist used in the composition to be administered to the subject in need thereof.

In the case where the administration is oral, the carrier is one that is acceptable for preparation of a dosage form suitable for oral administration. In the case where the administration is topical, the carrier is one that is acceptable for formulating a dosage form suitable for topical administration, one example being ocular administration, e.g. in the form of eye drops.

By the term “pharmaceutically acceptable carrier” it is meant any one of inert, non-toxic materials, which do not react with the A₃AR agonist and which can be added to formulations as diluents or carriers or to give form or consistency to the formulation.

An oral formulation may be in the form of a pill, capsule, in the form of a syrup, emulsion, an aromatic powder, and other various forms. The carrier is selected at times based on the desired form of the formulation. The carrier may also at times have the effect of the improving the delivery or penetration of the active ingredient to the target tissue, for improving the stability of the drug, for slowing clearance rates, for imparting slow release properties, for reducing undesired side effects etc. The carrier may also be a substance that stabilizes the formulation (e.g. a preservative), for providing the formulation with an edible flavor, etc. The carriers may be any of those conventionally used and is limited only by chemical-physical considerations, such as solubility and lack of reactivity with the A₃AR agonist, and by the route of administration. The carrier may include additives, colorants, diluents, buffering agents, disintegrating agents, moistening agents, preservatives, flavoring agents, and pharmacologically compatible carriers. In addition, the carrier may be an adjuvant, which, by definition are substances affecting the action of the active ingredient in a predictable way.

Typical examples of carriers suitable for oral administration comprise (a) suspensions or emulsions in an appropriate liquid such as Cremophor RH40, or methylcellulose (e.g. Methocel A4M Premium); (b) capsules (e.g. the ordinary hard- or soft-shelled gelatin type containing, for example, surfactants, lubricants, and inert fillers), tablets, lozenges (wherein the active substance is in a flavor, such as sucrose and acacia or tragacanth or the active substance is in an inert base, such as gelatin and glycerin), and troches, each containing a predetermined amount of the tragacanth as solids or granules; (c) powders; (d) solution, typically when combined with a solubilizing enhancing agent; (e) liposome formulation; and others.

One non limiting example for an oral administration form of the A₃AR agonist, IB-MECA includes the following ingredients and amounts formulated in the form of tablets:

TABLE 1
IB-MECA Tablets

| | Ingredient | Amount (mg) |
|---|---|---|
| Intragranular | IB-MECA | 1.000 |
| | Pregelatinized Starch | 10.00 |
| | Croscarmellose Sodium | 2.000 |
| | Lactose Monohydrate 310 | 64.25 |
| | Microcrystalline Cellulose | 20.00 |
| Extragranular | Croscarmellose Sodium | 2.000 |
| | Magnesium Stearate | 0.7500 |
| | Total | 100.00 |
| Coating | Opadry White | 3.000 |
| | Total | 103.0 |

A topical formulation may be in any form suitable for topical administration, including, without being limited thereto, an ophthalmic emulsion or solution (e.g. eye drops), an ophthalmic gel or an ophthalmic ointment or oily lotion. Topical administration of the A₃AR agonist also comprises the use of ophthalmic patches carrying the A₃AR agonist in a suitable drug containing layer and to be placed on top of the eyelid as well as to Ocular inserts which are devices containing the A₃AR agonist and placed into the inferior or superior conjunctival sacs (see for example WO0059420).

Eye drops may be prepared by suspending A₃AR agonist in a sterile aqueous solution such as saline, buffering solution etc., or by combining powder compositions to be dissolved before use. It is noted that as IB-MECA is not water soluble, when preparation a liquid formulation comprising IB-MECA, it may be require the use of emulsifiers, surfactants, slubilizing enhancing agents etc., in order to keep IB-MECA in the solution.

Other additives may be included in the eye drops such as isotonizing agents (e.g., sodium chloride, etc.), buffer agent (e.g., boric acid, sodium monohydrogen phosphate, sodium dihydrogen phosphate, etc.), preservatives (e. g., benzalkonium chloride, benzethonium chloride, chlorobutanol, etc.), thickeners (e. g., saccharide such as lactose, mahnitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, etc.).

Eye ointments may be prepared by mixing A₃AR agonist into a base. Examples of the base for eye ointment include petrolatum, selen 50, Plastibase, macrogol, etc., but not limited thereto.

Some exemplary ophthalmic viscosity enhancers that can be used in the present formulation include: carboxymethyl cellulose sodium; methylcellulose; hydroxypropyl cellulose; hydroxypropylmethyl cellulose; hydroxyethyl cellulose; polyethylene glycol 300; polyethylene glycol 400; polyvinyl alcohol; and providone.

Some natural products, such as veegum, alginates, xanthan gum, gelatin, acacia and tragacanth, may also be used to increase the viscosity of ophthalmic solutions.

A tonicity is important because hypotonic eye drops cause an edema of the cornea, and hypertonic eye drops cause deformation of the cornea. The ideal tonicity is approximately 300 mOsM. The tonicity can be achieved by methods described in Remington: The Science and Practice of Pharmacy, known to those versed in the art.

Additional administration routes may include, without being limited thereto, or parenteral administration (including subcutaneous, intramuscular and intravenous, intraarterial, intraperitoneally and intranasal) and others.

As used herein, the forms “a”, “an” and “the” include singular as well as plural references unless the context clearly dictates otherwise. For example, the term “an A₃AR agonist” includes one or more compounds which are capable of specifically binding to the A₃AR, thereby fully or partially activating said receptor.

Further, as used herein, the term “comprising” is intended to mean that the composition include the recited active agent, i.e. A₃AR agonist, but not excluding other elements, such as physiologically acceptable carriers and excipients as well as other active agents. The term “consisting essentially of” is used to define compositions which include the recited elements but exclude other elements that may have an essential significance on treatment of uveitis. “Consisting of” shall thus mean excluding more than trace elements of other elements. Embodiments defined by each of these transition terms are within the scope of this invention.

Further, all numerical values, e.g. when referring the amounts or ranges of the elements constituting the composition comprising the A₃AR agonist as an active ingredient, are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DESCRIPTION OF SOME NON-LIMITING EXAMPLES

### Effect of IB-MECA on the Development of Uveitis

### Materials & Methods

The A₃AR agonist that was used was a clinical grade of the compound known generically as 1-Deoxy-1-[6-[[(3-iodophenyl)methyl]amino]-9H-purine-9-yl]-N-methyl-D-ribofuranuronamide or as N⁶-(3-iodobenzyl)-adenosine-5′-N-methyluronamide (IB-MECA), that was synthesized for Can-Fite BioPharma, under good clinical practice (GMP) by Albany Molecular Research Inc, Albany, N.Y., USA. A stock solution of 10 μM of IB-MECA was prepared in dimethylsulfoxide (DMSO) and further dilutions were made in RPMI medium.

Experimental acute uveitis (EAU) was induced by immunizing C57BL/6j mice subcutaneously in the thighs and base of the tail with an emulsion of the retinal antigen interphotoreceptor retinoid-binding protein (IRPB, 200 μg per mouse) in incomplete Freund's adjuvant supplemented with Mycobacterium tuberculosis H37RA to 2.5 mg/ml. In addition, Pertussis toxin (300 ng/mouse) was injected intraperitoneally.

Oral treatment with IB-MECA (10 μg/kg per oz (Patent Office), twice daily) was initiated on day 7 after immunization. Disease intensity was scored by funduscopy upon pupil dilatation on day 16 and 20 after immunization. Scores were assigned according to the following: 0—no change; 0.5—Trace. Few (1-2) very small. Peripheral focal lesions, minimal vasculitis/viritis; 1—mild vasculitis, <5 small focal lesions, <1 linear lesion; 2-Multiple (>5) chorioretinal lesions and/or infiltrations; severe vasculitis (large size, thick wall, infiltrations); few linear lesions (<5).

Upon study termination, freshly enucleated eyes were fixed in phosphate-buffered glutaraldehyde, stained with hematoxylin and eosin and subjected to pathological analysis. The histological severity was graded on a scale of 0-4 based on the degree of cell infiltration, vasculitis, granuloma formation, photoreceptor cell damage in retina and choroid and retinal detachment in the eye.

To explore the immunological effects of IB-MECA on the antigen-specific responses of T cells, an in vitro antigen-driven proliferation assay was performed. Drain lymph nodes (inguinal and iliac) were collected from the IRBP immunized mice, both from the vehicle and from the IB-MECA treated groups. The cells were cultured for 48 hours in the presence of graded doses of IRBP (0.2-20 μg/ml) and proliferation was evaluated by an ³[H]-thymidine incorporation assay.

### Results

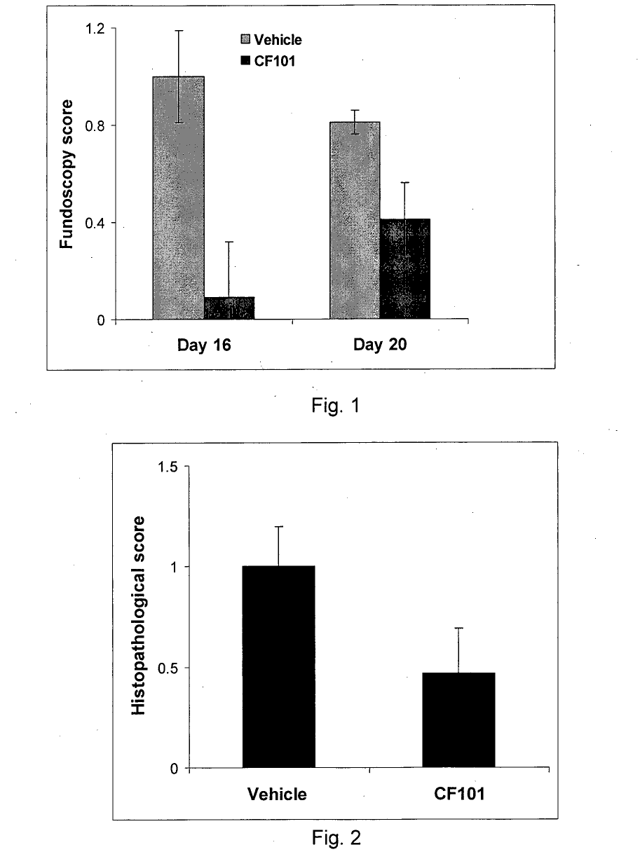

FIG. 1 shows that IB-MECA, identified in the figure by the code name CF101, treatment inhibited the fundoscopy score by 91% on day 16 and 49.4% on day 20 after immunization.

Further, FIG. 2 shows that IB-MECA, again identified in the figure by the code name CF101, treatment inhibited by 53% the pathological score in comparison to the vehicle-treated group, supporting the observations of the fundoscopy.

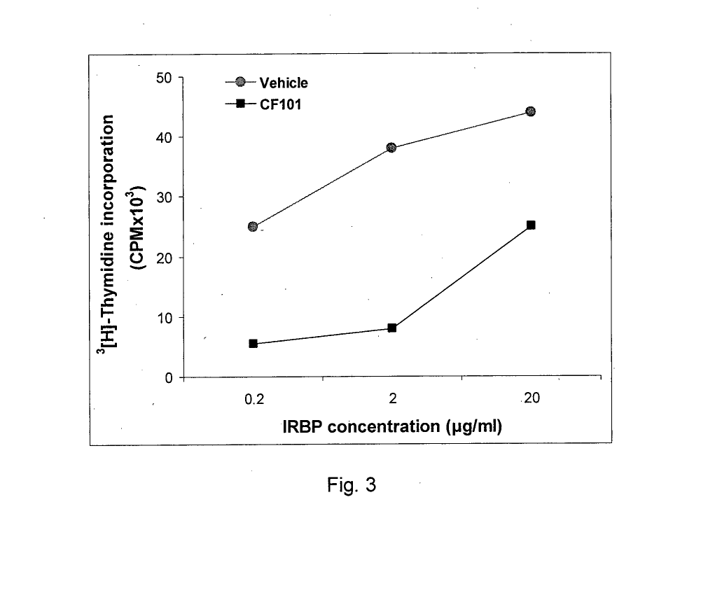

In cells derived from the vehicle-treated animals elevated T cell responses to IRBP were observed, while the cells derived from the IB-MECA treated animals exhibited a moderate response to the specific agonist, as shown in FIG. 3 (IB-MECA being identified in the figure by the code name CF101).

Taken together, IB-MECA reversed the development of the clinical and pathological scores of EAU and inhibited associated antigen-specific proliferative responses.

1-24. (canceled)

25. A method for the treatment of uveitis comprising administering a subject an amount of A₃ adenosine receptor (A₃AR) agonist, the amount being effective to treat or prevent uveitis.

26. The method of claim 25, wherein the A₃AR agonist is orally administered.

27. The method of claim 25, wherein the A₃AR agonist is administered twice a day.

28. The method of claim 25, wherein the A₃AR agonist is topically administered to said subject.

29. The method of claim 28, wherein the A₃AR agonist is topically administered to the subject's eye.

30. The method of claim 29, wherein the A₃AR agonist is administered to the subject's eye in the form of eye drops.

31. The method of claim 25, wherein the A₃AR agonist is selected from the group consisting of N⁶-2-(4-aminophenyl)ethyladenosine (APNEA), N⁶-(4-amino-3-iodobenzyl)adenosine-5′-(N-methyluronamide) (AB-ME CA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

32. The method of claim 31, wherein the A₃AR agonist is IB-MECA.
